(19) United States
(12) Patent Application Publication
(10) Pub. No.: US 2006/0129079 A1
(43) Pub. Date: Jun. 15, 2006

(54) Therapeutic device

(76) Inventor: Kevin Walter Outred, Kingsley (AU)

Correspondence Address: KENYON & KENYON LLP, 1500 K STREET N.W., SUITE 700, WASHINGTON, DC, 20005, US

(21) Appl. No.: 11/290,436

(22) Filed: Dec. 1, 2005

(30) Foreign Application Priority Data
Dec. 9, 2004 (AU) 2004-907114

(51) Int. Cl. A61F 5/00; A61F 13/06; A61F 13/00

(52) U.S. Cl. 602/21; 602/5; 602/2; 602/61

(57) ABSTRACT

The present invention provides a therapeutic device for a subject having inflammation of joints or tendons, the device comprising: (1) an elastic glove of thermotherapeutic material, (2) a pocket extending longitudinally along the palmar region of the glove, and (3) a brace which can be removably located in the pocket, wherein in use the brace is located in the pocket such that the palm is maintained at an acute angle relative to the longitudinal axis of the wearer's forearm. Preferably, the elastic glove is made of thermotherapeutic material to retain the wearer's body head and stimulate blood circulation and tissue healing. The brace is typically removably located in the pocket by sliding the brace through an opening in the pocket.

### FIELD OF THE INVENTION

The present invention relates to a therapeutic device. In particular it relates to a therapeutic glove for use by a person having inflammation or other disorder of the joints of the hand and fingers. More particularly the present invention relates to a therapeutic glove for use in the treatment of arthritis of the hand.

### BACKGROUND OF THE INVENTION

In this specification where a document, act or item of knowledge is referred to or discussed, this reference or discussion is not an admission that the document, act or item of knowledge or any combination thereof was at the priority date, publicly available, known to the public, part of the common general knowledge or known to be relevant to an attempt to solve a problem with which this specification is concerned.

While the present invention will be described with particular reference to rheumatoid arthritis the invention is not so limited but relates more generally to other arthritic disorders and inflammation of the hand joints or tendons including tendonitis and injuries caused by repetitive strain.

Arthritis is a common term applied to more than 100 different diseases that are characterised by inflammation of a joint. One of these diseases, perhaps the most common is the chronic disease known as rheumatoid arthritis. Rheumatoid arthritis occurs when the body's immune system attacks tissues that make up joints, principally affecting the synovium, the membrane that lines and lubricates a joint. As a result, the bones rub together, causing pain, swelling, and joint stiffness.

Of the 206 bones in the human body, over 50 are in the hand (including the wrist area of the hand) so there is a comparatively high probability of rheumatoid arthritis occurring in hand joints. Symptoms of arthritis include pain and swelling about the joint particularly with grasp and pinch. These symptoms may appear the first thing in the morning and be present for a half hour or so before the thumb “loosens up.” The symptoms often subside throughout the middle of the day, only to return with a “dull aching” type of pain towards the end of the day or after vigorous use. A “bump” may appear at the joint, due to the shifting of the joint bones as ligaments are loosened by swelling of adjacent tissue.

Treatment of arthritic conditions depends upon the symptoms and stage of the disease. For more severe symptoms, surgery may be necessary. For mild-to-moderate symptoms of pain and swelling, treatment consists primarily of anti-inflammatory medication, rest, appropriate exercise, splinting, and education.

Many different devices have been developed to treat rheumatoid arthritis. One such device is described in WO 94/01066 (Conran) and consists of a wrist brace comprising a glove covering the wearer's hand and forearm with a support means extending longitudinally in the glove to support the wrist. The glove can be opened by a fastening means such as a zipper or Velcro strip which thus allows access to the interior of the glove so that the support means can be put in position or removed from the device.

It is often difficult to strike a balance between appropriate levels of exercise and rest for an arthritic joint. Splinting is a popular treatment for arthritic joints and the restriction of movement and support provided by a splint helps to minimise pain. For example, U.S. Pat. No. 1,469,315 (Hansard) describes a device consisting of a glove having a pair of splints located on the back and front of a hand an forearm to prevent forward and backward movement, enabling only side to side movement of the hand relative to the arm. However restricting movement even for a limited period can cause joints to stiffen and weakens supporting muscles.

Restricting joint movement also reduces blood circulation to tissues, causing further tissue degeneration. Accordingly treatment for arthritis suffers commonly includes removal of a splint so that the affected joint can be soaked in hot water to maintain circulation and avoid ‘pooling’ of blood.

Accordingly there is a need for a device having wide applicability, which not only minimises pain by providing appropriate support and restriction of movement in a joint but which minimises further tissue degeneration or more preferably, promotes tissue recovery.

### SUMMARY OF THE INVENTION

The present invention provides a therapeutic device for a subject having inflammation of joints or tendons, the device comprising:

(1) an elastic glove of thermotherapeutic material,

(2) a pocket extending longitudinally along the palmar region of the glove, and

(3) a brace which can be removably located in the pocket,

wherein in use the brace is located in the pocket such that the palm is maintained at an acute angle relative to the longitudinal axis of the wearer's forearm. Preferably, the brace is removably located in the pocket by sliding the brace through an opening in the pocket.

The thermotherapeutic material retains the wearer's body heat, thus stimulating blood circulation and tissue healing. Typically the elastic glove has an upper surface and a lower surface, both of thermotherapeutic material. In use the upper surface of the elastic glove lies adjacent the back or dorsal surface of the hand and the lower surface is adjacent the palm. The upper and lower surfaces may be integral. For example the upper and lower surfaces may be sewn or adhered together to form the glove.

The upper surface and lower surface of the elastic glove may be of the same, or different thermotherapeutic material. For example the thermotherapeutic material may be of the type sold under the trade mark Thermoskin™. Typically the thermotherapeutic material will comprise 3 layers, that is, (1) a durable outer fabric, (2) a non-porous rubber material and (3) a knitted inner fabric such as that sold under the trade mark Trioxon®. When adjacent a wearer's skin, Trioxon has the advantage of allowing the skin to ventilate while absorbing perspiration, thus allowing the skin to remain comfortable and dry for relatively long periods.

Those suffering advanced rheumatoid arthritis often have difficulty gripping and keeping hold of objects due to deformation of joints and loss of muscle tone. Accordingly, in another preferred embodiment the lower surface of the elastic glove comprises a non-slip coating or outer layer that assists the wearer to maintain their grip on objects. For example, the lower surface may comprise thermotherapeutic material coated with synthetic rubber having a raised tread to improve the grip characteristics.

It is important that the glove has sufficient elasticity to provide support for the wearer's hand yet still allows some movement so that the joints do not stiffen and further weaken the hand muscles.

The elastic and thermotherapeutic characteristics of the glove stimulate blood flow to the wearer's hand in contrast to many of the devices of the prior art that hold the hand rigid so that blood flow is restricted and the natural recuperative functions of the body are inhibited.

Typically the elastic glove fits over the hand of the wearer in the manner of a gauntlet.

It may enclose the fingers or alternatively may leave part of the fingers exposed. For example it may leave exposed part or all of the distal, middle or proximal phalanx bones, in the manner of a ‘Fagan’ glove.

Preferably it does not extend much further than the lunoid or scaphoid bone, thus leaving wrist movement relatively unimpeded.

Preferably the elastic glove custom fits the wearer's hand and can simply be pulled on and off without the need for adjustment of size or shape. Alternatively the elastic glove may be adjustable particularly if, as in cases of severe arthritis, the wearer's hand periodically swells due to inflammation or other causes. For example, the elastic glove may include adjustable straps or tapes such as the hook and loop fastener known as Velcro™. Typically the adjustable straps or tapes would be located adjacent the metacarpals or the scaphoid bone in the wrist area of the hand.

Typically the brace consists of a strip of rigid material such as metal or plastic that has two integral limbs joined to form an obtuse angle of between about 155 and 140°. In use one limb of the brace lies adjacent the wrist bones while the other limb lies adjacent the metacarpals, preferably in the centre of the wearer's palm adjacent the 3rd metacarpal bone. The obtuse angle between the two limbs of the brace keeps the metacarpals at an angle of about 25 to 40° relative to the bones of the wrist and forearm.

The therapeutic device of the present invention is therefore distinguished from similar inventions of the prior art that extend beyond the wrist region of the wearer's hand and brace the bones of the wearer's forearm.

The pocket is generally elongate, commensurate with the length and width of the brace and is made of any convenient material that is sufficiently robust to maintain the brace in position. As the pocket is not adjacent the user's skin it is not necessary that it be made of thermotherapeutic material. Typically the opening in the pocket is located adjacent the wrist bones of the hand.

### DRAWINGS

The invention will now be further described with reference to the following non-limiting drawings:

FIG. 1a shows a perspective view of a hand located in a therapeutic device according to one embodiment of the present invention,

FIG. 1b shows a plan view of the dorsal surface of the therapeutic device of FIG. 1a,

FIG. 2 shows a plan or ‘x-ray’ view of the bones of a hand located in a therapeutic device according to another embodiment of the present invention, and

FIG. 3 shows a brace suitable for use in the therapeutic device of FIG. 1 or FIG. 2.

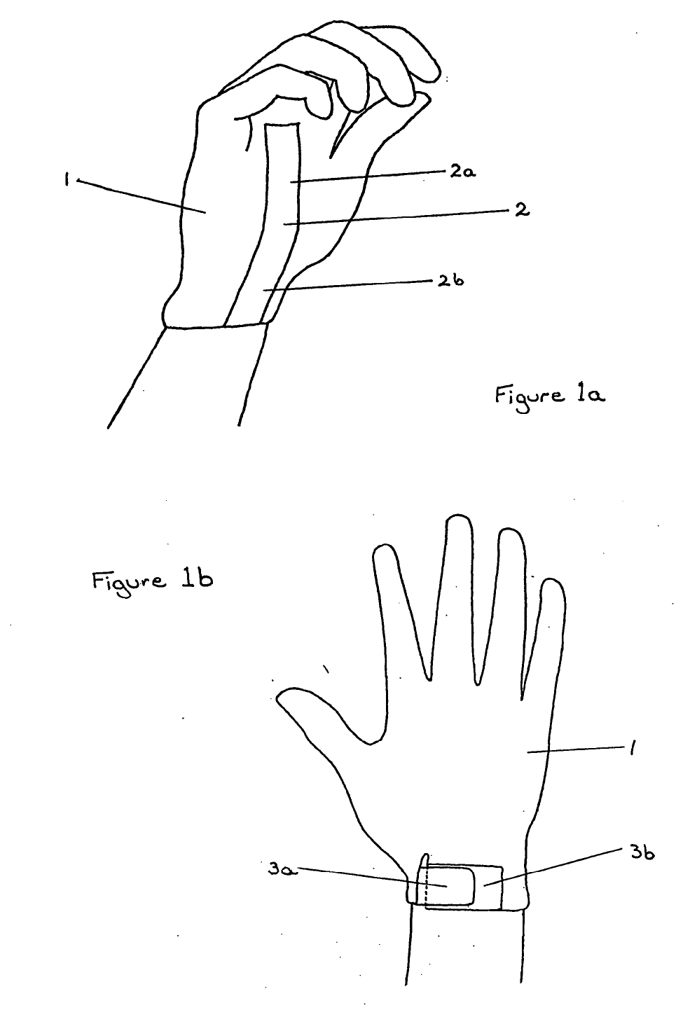

FIG. 1a shows a hand located in a therapeutic device (1) according to one embodiment of the present invention. The therapeutic device (1) is made of Thermoskin™ thermotherapeutic material and entirely encloses the wearer's fingers and palm to the level of the wrist. The pocket (2) runs longitudinally and centrally along the palm of the hand, the opening of the pocket being adjacent the wrist. As shown in this view the brace has 2 limbs (2a, 2b) that meet at an obtuse angle. The brace can readily be slid out of the pocket when, for example, the elastic glove needs to be washed.

FIG. 1b shows the dorsal side of the device shown in FIG. 1a. The elastic glove is adjustable and this view clearly shows a Velcro fastener consisting of a tape of hooks (3a) and a tape of loops (3b). The tape of hooks (3a) can be moved to any convenient position relative to the tape of loops (3b) in order to tighten or loosen the fit of the elastic glove around the wearer's wrist. It will be apparent to those skilled in the art that it would also be possible to use a strap and buckle fastener or the like instead of the Velcro fastener.

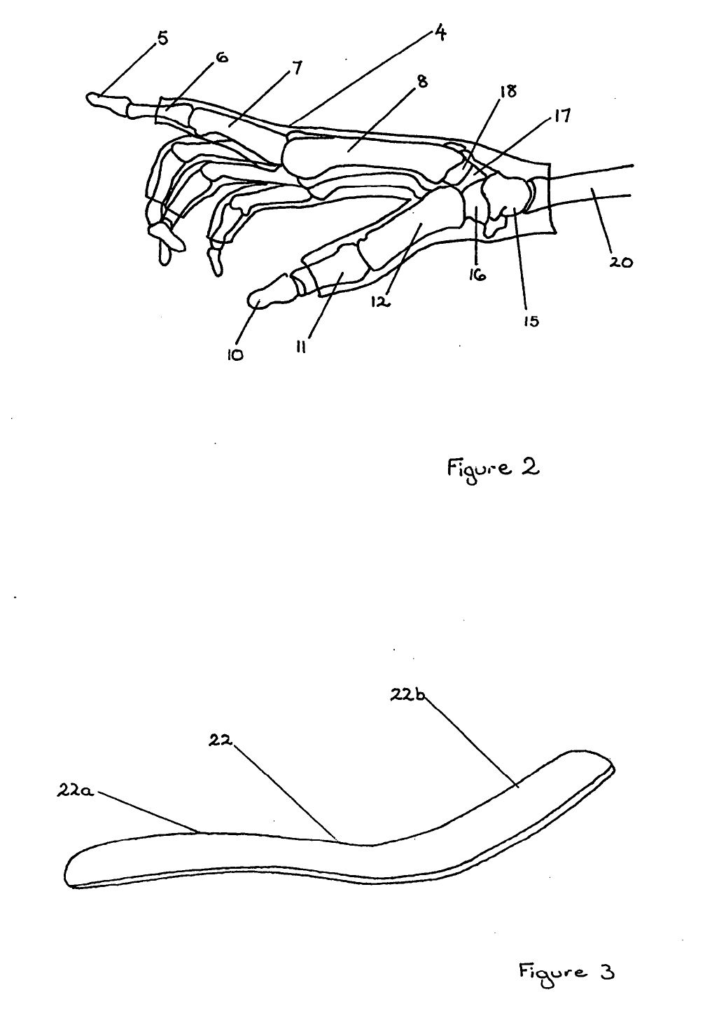

FIG. 2 shows the location of some of the bones of the hand in another embodiment of the therapeutic device (4) of the present invention. In this view the 2nd distal (5), 2nd medial (6), 2nd proximal (7) and 2nd metacarpal (8) bones of the forefinger can be seen, along with the 1st distal (10), 1st proximal (11) and 1st metacarpal (12) bones of the thumb. Eight bones make up the wrist portion of the hand (the lunate, scaphoid, trapezium, trapezoid, capitate, hamate, pisiform and triquetrum bones) of which only the scaphoid (15), trapezium (16), trapezoid (17) and capitate (18) bones can be seen in this view. In this embodiment the therapeutic device does not enclose the wearer's fingers but leaves the distal bones, and part of the medial and 1 st proximal bones exposed. The elastic glove encloses the rest of the hand, terminating at the wrist, adjacent the top of the ulna (20) and radius (not shown).

FIG. 3 shows a brace (22) suitable for use in the therapeutic device of FIG. 1 or FIG. 2, the brace (22) consisting of a thin strip of metal with rounded corners, having a first limb (22a) and a second limb (22b) so that the brace is in the form of a wide ‘V’. The longitudinal axes of the two limbs form an obtuse angle of about 150°, which in use keeps the wearer's metacarpals at an approximately 30° angle to the bones of the forearm.

The word ‘comprising’ and forms of the word ‘comprising’ as used in this description does not limit the invention claimed to exclude any variants or additions.

Modifications and improvements to the invention will be readily apparent to those skilled in the art. Such modifications and improvements are intended to be within the scope of this invention.

The claims defining the invention are as follows:

1. A therapeutic device for a subject having inflammation of joints or tendons, the device comprising:
(1) an elastic glove of thermotherapeutic material,
(2) a pocket extending longitudinally along the palmar region of the glove, and
(3) a brace which can be removably located in the pocket,
wherein in use the brace is located in the pocket such that the palm is maintained at an acute angle relative to the longitudinal axis of the wearer's forearm. Preferably, the brace is removably located in the pocket by sliding the brace through an opening in the pocket.

2. A therapeutic device according to claim 1 wherein the elastic glove has an upper surface and a lower surface which surfaces are integral.

3. A therapeutic device according to claim 1 wherein the elastic glove has an upper surface and a lower surface, both comprised of thermotherapeutic material.

4. A therapeutic device according to claim 1 wherein the elastic glove has an upper surface and a lower surface and each of the upper surface and lower surface comprises (1) a durable outer fabric, (2) a non-porous rubber material and (3) a knitted inner fabric.

5. A therapeutic device according to claim 2 wherein the lower surface of the elastic glove comprises a non-slip coating or outer layer that assists the wearer to maintain their grip on objects.

6. A therapeutic device according to claim 1 wherein the elastic glove fits over the hand of the wearer and encloses one or more of the wearer's fingers.

7. A therapeutic device according to any claim 1 wherein the elastic glove does not extend further than the wearer's lunoid bone.

8. A therapeutic device according to claim 1 wherein the fit of the elastic glove to the wearer is adjustable.

9. A therapeutic device according to claim 1 wherein the fit of the elastic glove can be adjusted by straps or tapes.

10. A therapeutic device according to claim 1 wherein the brace consists of a strip of rigid material having two integral limbs joined to form an obtuse angle of between about 155 and 140°.

11. A therapeutic device according to claim 1 wherein the brace consists of two integral limbs wherein in use, one limb of the brace lies adjacent the wrist bones while the other limb lies adjacent the metacarpals.

12. A therapeutic device according to claim 1 wherein in use, the brace keeps the wearer's metacarpals at an angle of about 25 to 40° relative to the bones of the wrist and forearm.
